(19) United States
(12) Patent Application Publication
(10) Pub. No.: US 2025/0371714 A1
(43) Pub. Date: Dec. 4, 2025

(54) SEMI-SUPERVISED IMAGE SEGMENTATION FOR MEDICAL DECISION MAKING

(71) Applicant: NEC Laboratories America, Inc., Princeton, NJ (US)

(72) Inventors: Renqiang Min, Princeton, NJ (US); Eric Cosatto, Red Bank, NJ (US); Kai Li, Plainsboro, NJ (US)

(21) Appl. No.: 19/219,310

(22) Filed: May 27, 2025

Related U.S. Application Data
(60) Provisional application No. 63/652,285, filed on May 28, 2024.

(51) Int. Cl. G06T 7/194; G06T 7/00; G16H 50/20

(52) U.S. Cl. CPC G06T 7/194; G06T 7/0012; G16H 50/20; G06T 2207/20081; G06T 2207/20112; G06T 2207/30024

(57) ABSTRACT

Methods and systems for image segmentation include initializing a student model and a teacher model using a labeled dataset. An initial mask is generated for an unlabeled image using the teacher model. The initial mask is refined to generate a refined mask using a pretrained foundation model. The student model is tuned using the unlabeled image and the refined mask as a pseudo-ground truth label. The teacher model is updated using the tuned student model.

### RELATED APPLICATION INFORMATION

This application claims priority to U.S. Patent Application No. 63/652,285, filed on May 28, 2024, incorporated herein by reference in its entirety.

### BACKGROUND

### Technical Field

The present invention relates to image analysis and, more particularly, to image segmentation.

### Description of the Related Art

Image segmentation helps to extract meaningful information from complex imaging datasets. Delineating structures within an image can help to identify objects of interest and can be important for effective downstream processing. However, image segmentation can be challenging in domains where labeled data is limited. This is particularly pronounced in label-deficient domains, where the availability of annotated images falls short. Manual annotation of images is time-consuming and resource intensive. As a result, the limited availability of labeled data is a bottleneck to the development of high-performance segmentation models in new domains.

### SUMMARY

A method for image segmentation includes initializing a student model and a teacher model using a labeled dataset. An initial mask is generated for an unlabeled image using the teacher model. The initial mask is refined to generate a refined mask using a pretrained foundation model. The student model is tuned using the unlabeled image and the refined mask as a pseudo-ground-truth label. The teacher model is updated using the tuned student model.

A system for image segmentation includes a hardware processor and a memory that stores a computer program. When executed by the hardware processor, the computer program causes the hardware processor to initialize a student model and a teacher model using a labeled dataset, to generate an initial mask for an unlabeled image using the teacher model, to refine the initial mask to generate a refined mask using a pretrained foundation model, to tune the student model using the unlabeled image and the refined mask as a pseudo-ground-truth label, and to update the teacher model using the tuned student model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of a process for training a segmentation model using a student model/teacher model process that takes advantage of a pretrained foundation model to refine masks in a semi-supervised training approach, in accordance with an embodiment of the present invention;

FIG. 2 is a block/flow diagram of a method for training a segmentation model using an iterative process that creates pseudo-ground-truth labels for an unlabeled dataset, in accordance with an embodiment of the present invention;

FIG. 3 is a block/flow diagram of a method for training and using a segmentation model, in accordance with an embodiment of the present invention;

FIG. 4 is a block diagram of a healthcare facility where image segmentation is used to aid in medical decision making, in accordance with an embodiment of the present invention;

FIG. 5 is a block diagram of a computing device that can be used to train and use a segmentation model, in accordance with an embodiment of the present invention;

FIG. 6 is a diagram of an exemplary neural network architecture that can be used to implement part of a segmentation model, in accordance with an embodiment of the present invention; and

FIG. 7 is a diagram of an exemplary deep neural network architecture that can be used to implement part of a segmentation model, in accordance with an embodiment of the present invention. and

### DETAILED DESCRIPTION OF PREFERRED EMBODIMENTS

Semi-supervised segmentation can be used to provide a segmentation model based on relatively few labeled images and a large number of unlabeled images. Since only a few images need to be annotated, the cost of labeling is reduced significantly. A large, multimodal foundation model may be pre-trained on extensive datasets, encompassing a variety of different imaging modalities, and can learn intricate feature representations. Such models provide a capacity to capture complex patterns and semantic information, enabling them to generalize well even in the absence of an abundance of labeled training data.

In particular, a multimodal foundation model can be used to produce accurate masks given an input image and prompts such as points, bounding boxes, and masks. A standard model may first be trained with labeled images. The standard model may then be used to make predictions on unlabeled images. The predictions are used as a prompt for the multimodal foundation model to produce a more accurate mask. The refined mask may then be used as a pseudo-ground-truth to train an image segmentation model. Noise in the predicted labels may be reduced using a student-teacher structure, where the teacher model produces the initial mask and trains the student model with the pseudo-ground-truth mask. The teacher model may be updated as the exponential moving average of the student model.

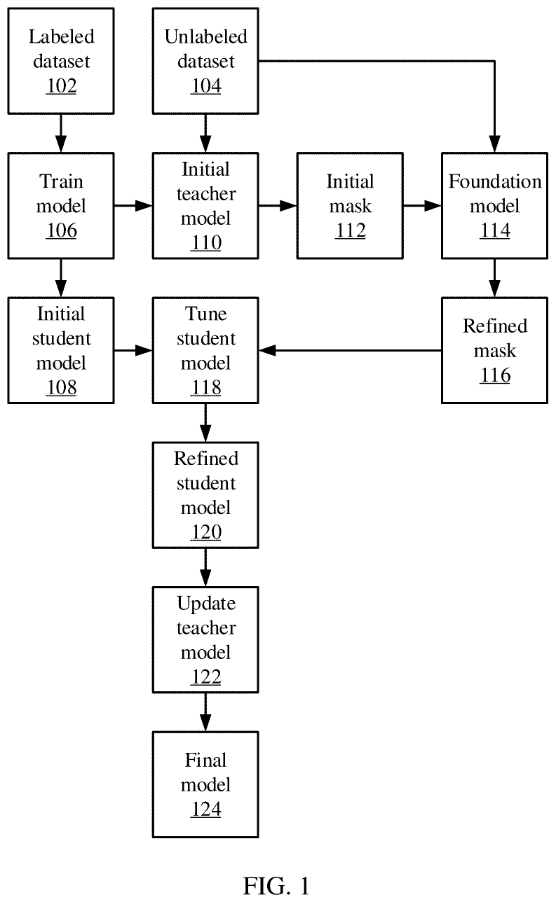

Referring now to FIG. 1, an overview of a training process for a segmentation model is shown. Labeled dataset 102 and unlabeled dataset 104 are provided, where the unlabeled dataset 104 may be substantially larger than the labeled dataset 102. The labeled dataset 102 may include images and corresponding mask labels that indicate how the image should be segmented. The unlabeled dataset 104 may include only images, with no corresponding mask.

An initial model training 106 is performed using the relatively limited labeled dataset 102. Any appropriate segmentation model architecture may be used to create an initial segmentation model, which is used to initialize the initial teacher model 110 (parameters θₜ) and the initial student model 108 (parameters θₛ). The initial teacher model 110 creates initial masks 112 from images of the unlabeled dataset 104.

A pretrained foundation model 114 is used to generate refined masks 116 from the initial masks 112. For example the Segmentation Anything Model (SAM) may be used as a pretrained multimodal foundation model. The use of the pretrained foundation model 114 helps to prevent the accumulation of noise in the pseudo-labels. The refined masks 116 are used as pseudo-ground-truth labels for the unlabeled dataset 104 in tuning 118 of the initial student model 108, for example using backpropagation to produce a refined student model 120. The teacher model may be updated 122 as an exponential moving average of the refined student model 120 to produce a final model 124. For example, the teacher model 110 may be updated as θₜ=αθₜ+ (1−α)θₛ, where a is a weighting hyperparameter that is close to 1. This approach to updating the teacher model 110, in contrast to backpropagation, keeps the teacher model 110 stable if a mask produced by the foundation model 114 is not reliable, thereby mitigating error accumulation. The final model 124 may then be used for segmentation on new input images.

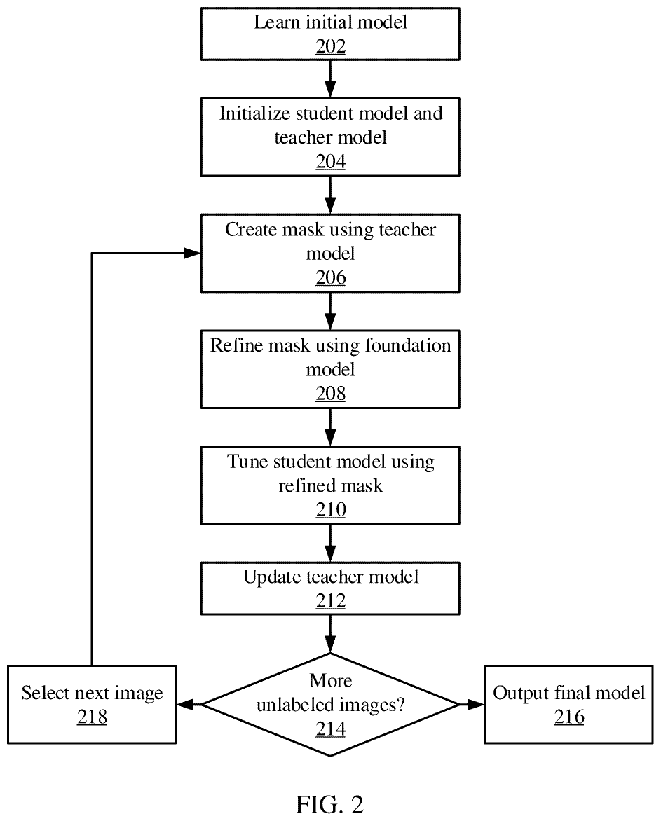

Referring now to FIG. 2, a method of training a segmentation model is shown. Block 202 learns an initial segmentation model using the labeled dataset 102. Block 204 initializes the initial student model 108 and the initial teacher model 110, for example by copying the parameters of the initial segmentation model. Block 206 creates an initial mask 112 using the teacher model. Block 208 then refines the mask using the foundation model 114 to create refined mask 116.

Block 210 tunes the student model using the refined mask 116, generating a refined student model 120. The refined mask 116 is used as a pseudo-ground-truth label for backpropagation. Block 212 then uses the refined student model 120 to update the teacher model.

Block 214 determines whether there are additional unlabeled images in the unlabeled dataset 104. If so, block 218 selects a next image from the unlabeled dataset 104 and processing returns to block 206 for a new pseudo-ground-truth label. If the unlabeled dataset 104 has been exhausted, or some other stopping criterion has been reached (e.g., convergence), then block 216 outputs the updated teacher model as a final model 124.

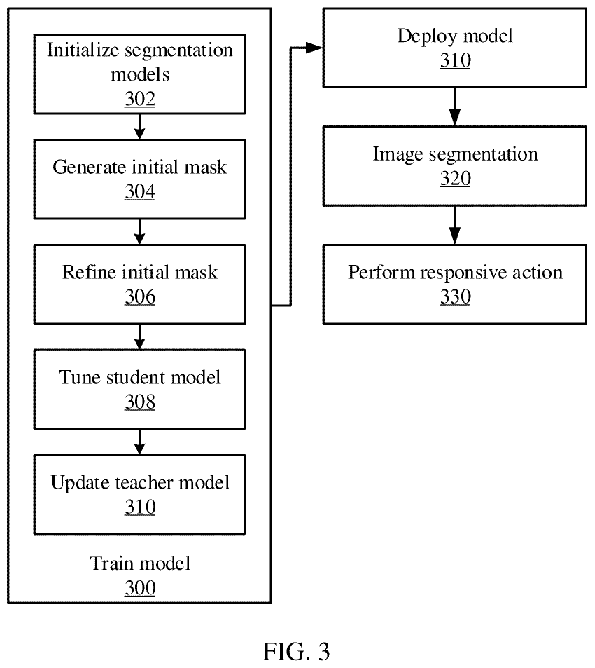

Referring now to FIG. 3, a method for training and using a segmentation model is shown. Block 300 trains the model as above, using an iterative process to generate pseudo-ground-truth labels for an unlabeled dataset. Block 302 initializes the student model 108 and the teacher model 110. Block 304 generates the initial mask using the teacher model 110 and then block 306 refines the initial mask using the pretrained foundation model. Block 308 tunes the student model 308 using the refined mask as a pseudo-ground-truth label for the unlabeled image. Block 310 then updates the teacher model using the tuned student model. This process repeats until a final teacher model is output as the trained segmentation model.

Block 310 deploys the trained segmentation model to a target system, where new images will be processed. In some cases, where the target system is the same as the system where training is performed, deployment 310 may be omitted. Deploying the trained model may include copying the parameters of the trained segmentation model to the target system.

Block 320 performs image segmentation at the target system. For example, the target system may obtain a new image and the trained model may be used to perform image segmentation. The output of the trained model may include a mask that indicates a region of interest within the new image.

Block 330 then performs an action responsive to the image segmentation. In some embodiments, the new image may be an image of a tissue sample in a medical context, where the mask indicates locations of a particular type of tissue (e.g., tumor cells). Block 330 may thus include performing diagnosis and treatment of a disease using the mask.

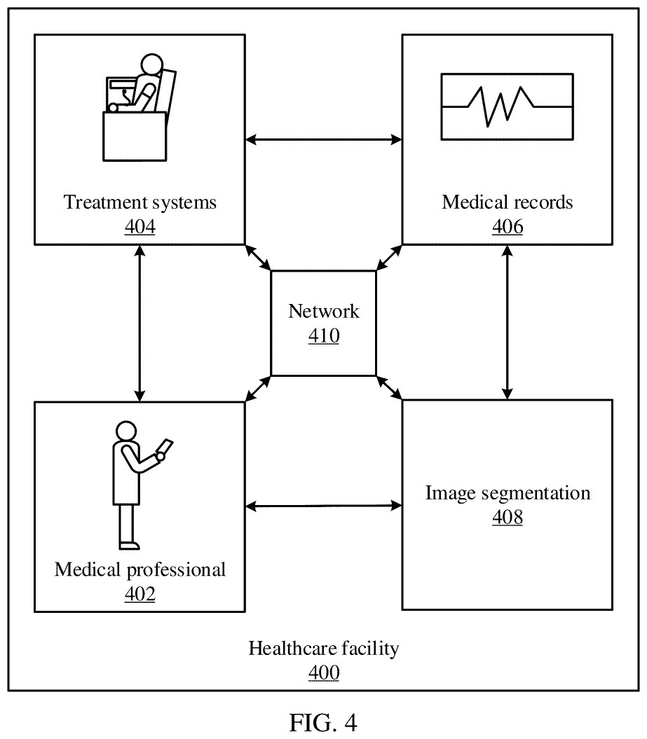

Referring now to FIG. 4, a diagram of a healthcare facility is shown. Referring now to FIG. 4, a diagram of therapy generation is shown in the context of a healthcare facility 400. Image segmentation 408 may be used to diagnose a disease, for example using tissue sample images from medical history 406, which can be used to generate a custom treatment for a patient. The image segmentation 408 may be used to identify, for example, the presence and extent of tumor cells in the tissue sample, which may be used to generate a treatment responsive to a patient's medical condition.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Medical professionals 402 may use image segmentation 408 to provide customized healthcare that is tailored to the patient's needs. For example, the medical professionals 402 may use image segmentation 408 to diagnose a cancer or other disease and so select a tailored treatment.

The different elements of the healthcare facility 400 may communicate with one another via a network 410, for example using any appropriate wired or wireless communications protocol and medium. Thus the image segmentation 408 can be used to diagnose and to design a treatment that targets a patient's specific condition, for example using test results and medical records 406. The treatment systems 404 may be used to generate and administer a therapy based on image segmentation 408.

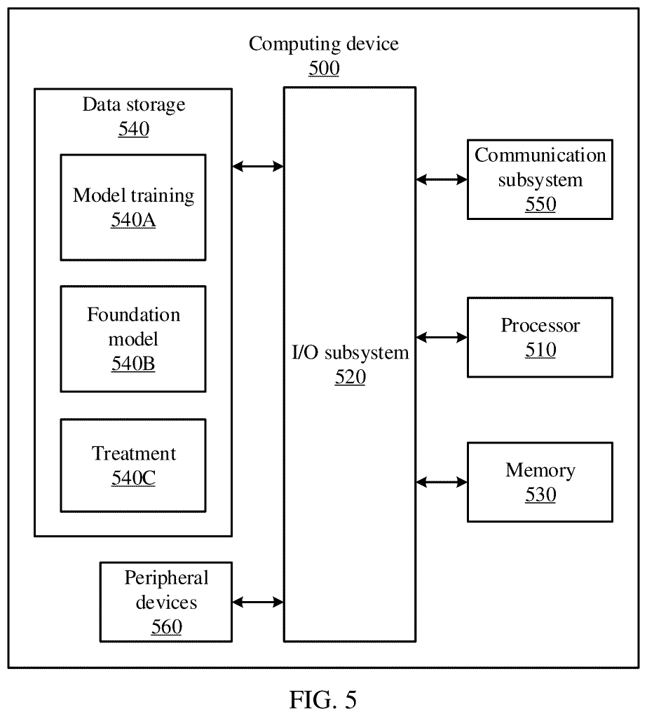

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for training a segmentation model, 540B to implement a pretrained multimodal foundation model, and/or 540C for generating a treatment. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

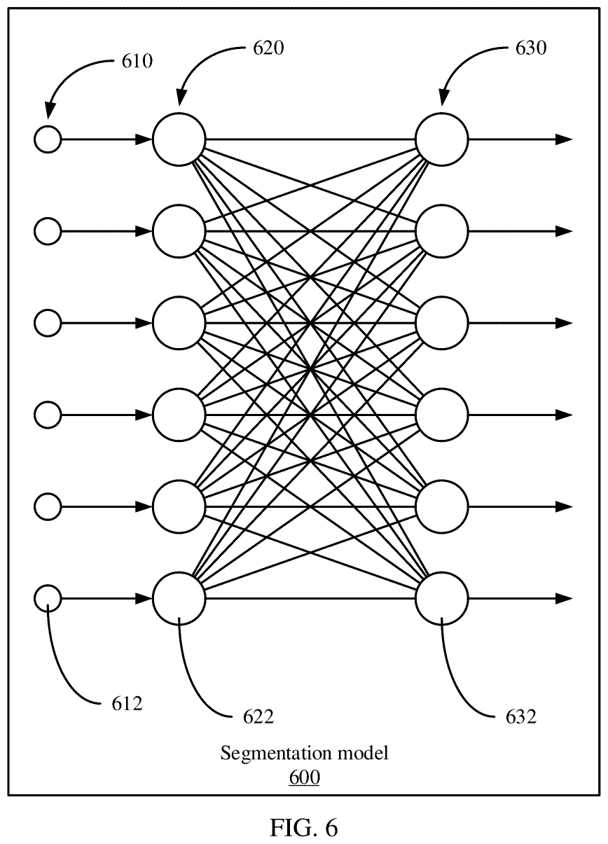

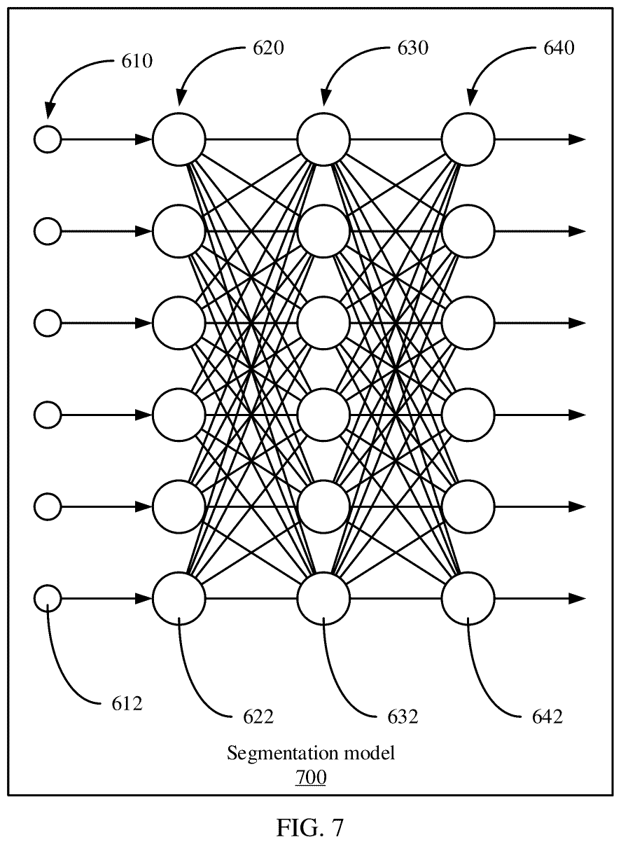

Referring now to FIGS. 6 and 7, exemplary neural network architectures are shown, which may be used to implement parts of the present models, such as the segmentation model 600/700. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for image segmentation, comprising:
initializing a student model and a teacher model using a labeled dataset;
generating an initial mask for an unlabeled image using the teacher model;
refining the initial mask to generate a refined mask using a pretrained foundation model;
tuning the student model using the unlabeled image and the refined mask as a pseudo-ground-truth label; and
updating the teacher model using the tuned student model.

2. The method of claim 1, further comprising repeating the generating, refining, tuning and updating for additional unlabeled images of an unlabeled dataset.

3. The method of claim 2, wherein updating the teacher model includes an exponential moving average of the tuned student model.

4. The method of claim 3, wherein the exponential moving average is expressed as:
$${θ}_{t} = {αθ}_{t} + ( 1 - α ) {θ}_{s}$$
where θₜ is the teacher model, θₛ is the student model, and a is a weighting hyperparameter.

5. The method of claim 2, wherein the unlabeled dataset is larger than the labeled dataset.

6. The method of claim 1, wherein the teacher model and the student model are machine learning models that accept an image as input and that output a segmentation mask.

7. The method of claim 1, wherein the labeled dataset includes images of tissue samples with labels that include masks indicating a cell type.

8. The method of claim 7, further comprising performing image segmentation on a new image using the updated teacher model.

9. The method of claim 8, further comprising performing a treatment action responsive to the image segmentation.

10. The method of claim 8, wherein the image segmentation is used for medical decision making.

11. A system for image segmentation, comprising:
a hardware processor; and
a memory that stores a computer program which, when executed by the hardware processor, causes the hardware processor to:
initialize a student model and a teacher model using a labeled dataset;
generate an initial mask for an unlabeled image using the teacher model;
refine the initial mask to generate a refined mask using a pretrained foundation model;
tune the student model using the unlabeled image and the refined mask as a pseudo-ground-truth label; and
update the teacher model using the tuned student model.

12. The system of claim 11, wherein the computer program further causes the hardware processor to repeat the generation, refinement, tuning and update for additional unlabeled images of an unlabeled dataset.

13. The system of claim 12, wherein the update of the teacher model includes an exponential moving average of the tuned student model.

14. The system of claim 13, wherein the exponential moving average is expressed as:
$${θ}_{t} = {αθ}_{t} + ( 1 - α ) {θ}_{s}$$
where θₜ is the teacher model, θₛ is the student model, and a is a weighting hyperparameter.

15. The system of claim 12, wherein the unlabeled dataset is larger than the labeled dataset.

16. The system of claim 11, wherein the teacher model and the student model are machine learning models that accept an image as input and that output a segmentation mask.

17. The system of claim 11, wherein the labeled dataset includes images of tissue samples with labels that include masks indicating a cell type.

18. The system of claim 17, wherein the computer program further causes the hardware processor to perform image segmentation on a new image using the updated teacher model.

19. The system of claim 18, wherein the computer program further causes the hardware processor to perform a treatment action responsive to the image segmentation.

20. The system of claim 18, wherein the image segmentation is used for medical decision making.
